(19) United States
(12) Patent Application Publication
(10) Pub. No.: US 2004/0052754 A1
(43) Pub. Date: Mar. 18, 2004

(54) Complexes of phosphate derivatives

(76) Inventors: Simon Michael West, Williamstown (AU); Robert J. Verdicchio, Succasunna, NJ (US); David Kannar, Belgrave South (AU)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(21) Appl. No.: 10/462,480

(22) Filed: Jun. 16, 2003

Related U.S. Application Data
(63) Continuation-in-part of application No. 10/416,774.
Application No. 10/416,774 is a 371 of international application No. PCT/AU01/01476, filed on Nov. 14, 2001.
(60) Provisional application No. 60/247,997, filed on Nov. 14, 2000.

(51) Int. Cl. A61K 7/075; A61K 7/08

(52) U.S. Cl. 424/70.23; 424/70.27; 424/70.28; 424/70.24

(57) ABSTRACT

There is provided a composition comprising the reaction product of:

(a) one or more phosphate derivatives of one or more hydroxylated actives; and

(b) one or more complexing agents selected from the group consisting of amphoteric surfactants, cationic surfactants, amino acids having nitrogen functional groups and proteins rich in these amino acids.

This application is a Continuation-In-Part application of U.S. patent application Ser. No. 10/416,774 filed as a National Phase Application on May 14, 2003 which claims the benefit of International Application No. PCT/AU01/01476 filed Nov. 14, 2001 which claims priority of U.S. Provisional Application No. 60/247,997 dated Nov. 14, 2000, the complete disclosures of which are incorporated herein by reference.

### FIELD OF THE INVENTION

The invention relates to complexes of phosphate derivatives. More particularly the invention relates to complexes of phosphate derivatives of hydroxylated active compounds.

### BACKGROUND OF THE INVENTION

In this specification, where a document, act or item of knowledge is referred to or discussed, this reference or discussion is not to be taken as an admission that the document, act or item of knowledge was at the priority date:

(a) part of common general knowledge; or

(b) known to be relevant to an attempt to solve any problem with which this specification is concerned.

Over the past century, quantitative structure activity relationships (QSAR) have evolved and predominated in medicinal chemistry research programs. QSAR methods generate mathematical models to describe biological function of drug formulations. Deriving a mathematical description of biological activity is characterized by two assumptions with respect to the relationship between the chemical structure and the biological potency of a compound. The first is that one can transform the chemical structure of a compound into numerical descriptors relevant to biological activity of a compound. The second establishes a quantitative relationship between these mathematical descriptors and potential biological activity.

The mathematical descriptors are usually either physiochemical, such as pKa or partition coefficient, or substructural, such as the presence or absence of functional groups, eg. CO₂R or SH, and assist the formulating chemist to improve the solubility of the biologically active compound.

This is recognized to revolve around fundamental strategies aimed to increase solubility and dissolution rate of drugs derived from dosage forms. Theoretically, these strategies make the drug more available for absorption, and involve techniques such as co-solvent addition, solid state manipulation and pro-drug modification.

Lipids as Carriers

A number of drugs are more lipid soluble rather than water soluble, therefore lipids have been the carrier of choice for such drugs. Lipids are selected as drug vehicles based on their digestibility. Surfactant and co-solvent addition can facilitate digestion by increasing solubilization within the intestine and formation of chylomicrons/VLDL by the enterocyte to improve lymphatic transport.

Lipid-based formulations, in particular, self-emulsifying drug delivery systems (SEDDS) and self micro-emulsifying drug delivery systems (SMEDDS) which utilize isotropic mixtures of triglyceride oils, non-surfactants and drugs, have been shown to overcome some of the barriers resulting in improved absorption characteristics and more reproducible plasma profiles of selected drugs.

SEDDS and SMEDDS can be filled into either soft or hard gelatine capsules, allowing rapid emulsification following release of the capsule contents and exposure to gentle agitation in an aqueous media. Following emulsification, the fine oil droplets (<5 μm in diameter) empty rapidly from the stomach and promote wide distribution of the lipophilic drug throughout the gastrointestinal tract. This fine droplet distribution increases surface area for the drug to partition into the intestine and should theoretically improve absorption.

Derivatisation

Another strategy to improve solubility is to derivatise the compound, also known as forming pro-drugs. A number of undesirable properties may preclude the use of potentially valuable drug drugs in clinical practice. Derivatisation has long been recognized as an important means of increasing efficacy and bioavailability of such drugs. Pro-drugs may be of limited value unless the pro-drug displays adequate stability, solubility, permeability and capability to revert to the parent compound once absorbed into the systemic circulation.

For example, one earlier attempt to address this problem involved forming covalent bonds with sugars and polyalcohols. However, further problems were created as the additional substituent must then be removed before drug activity is regenerated. For example, tocopherol polyethylene glycol succinate (TPGS) is being sold as a water soluble derivative of α-tocopherol. There are indications that this derivative is absorbed even when bile secretion is impaired however, the issue of hydrolysis of the ester linkage to succinate and metabolism of the resulting polyethylene glycol 1000 does not seem to have been addressed. It has not been established if and when the ester group hydrolyses. If the ester group does not hydrolyse then the tocopherol is not released and cannot act on the body. If the ester is hydrolysed then the next issue is whether the body can metabolise the polyethylene glycol by-product and dispose of it. If the body cannot metabolise the by-product then there could be a build up of by-product leading to side-effects. The TPGS product is also inconvenient and difficult to utilize clinically.

Limitations of Current Drug Solubilisation Strategies

Today, QSAR remains a useful tool to help discover, quantify and evaluate possible biological activity. However, QSAR has been criticized for not being able to effectively generate descriptors for three dimensional features, such as hydrophobicity and some electronic effects of drug interaction including hydrogen bonding. QSAR is also known to be inadequate in relation to describing various biological processes including gastrointestinal absorption, distribution, metabolism and excretion.

Development of lipid formulation strategies have also been helpful but only based upon the assumption that important biologically active compounds are passively absorbed and providing a dissolution gradient will improve absorption. This assumption is flawed and does not account for the possibility of active absorption. This delivery strategy therefore remains limited and cannot account for the fact that even after optimal formulation, absorption of poorly soluble nutrients from food is higher.

While ester derivatisation and solubilistion in SEDDS are known to improve lymphatic transport by the notion of forming small lipidic artificial chylomicrons, the methods are inefficient and probably more important to permit metabolism, rather than increasing transport of intact lipidic microstructures recognisable by transfer proteins. The use of alternative historic formulation strategies may therefore even restrict clinical utility of α-tocopherol and result in reduced efficacy.

Example of the Limitations of QSAR Formulation Approaches

Tocopherol (vitamin E) is a poorly absorbed, lipid soluble vitamin and chemically unstable due to oxidation of the phenolic group. The majority of natural tocopherol is currently extracted from soy oil distillate and presented as simple substituted esters—either succinate or acetate derivatives. While this is primarily undertaken to prevent oxidation of the phenolic group and enhance stability, derivatisation is also thought to improve lymphatic transport. There have been a number of attempts to enhance α-tocopherol acetate lymphatic transport via lipid formulation approaches. However despite some improvement, the extent of α-tocopheryl ester absorption after oral supplement administration is still poor and subject to large inter-patient variation. In contrast, dietary intake of vitamin E may result in a rapid and parallel increase in the content of α-tocopherol in blood plasma and erythrocytes.

Other drugs and nutrients are also subject to poor and variable absorption properties following current oral formulation strategies including phenytoin, vitamin A and CoQ₁₀, suggesting that physio-chemical factors other than dispersion, digestion and solubilisation control their bioavailability.

Transportation

In recent years it has become apparent that absorption across biological membranes of some pharmacologically active compounds eg: drugs and nutrients (vitamin E, ubiquinone, etc.), and endogenously important compounds such as phospholipids may be the limiting factor for bioavailability. As suggested such biological processes are difficult to describe mathematically as they are often multi dimensional. It is therefore proposed that gastrointestinal uptake and transport of many biologically active compounds is dependent on other transportation mechanisms.

Studies have shown that α-tocopherol phosphate is an effective antioxidant and capable of preventing hypoxanthine/xanthine oxidase induced oxidative damage. α-tocopherol phosphate is more water soluble than tocopherol or its succinate esters. These studies indicate that α-tocopherol phosphate not only improves chylomicron formation but also improves tissue penetration.

The art of efficient drug delivery therefore requires that the drug be not only soluble in the aqueous biological medium but in an appropriate form to permit transport of either individual drug molecules or very small aggregates of the drug molecules. This aim may be difficult to realize with drugs that are lipid soluble and not significantly water soluble. Such drug molecules have hydrophobic regions that form large aggregates in the high dielectric constant water rich medium where transport occurs. As a result, there have been investigations to discover a drug delivery system which increases the water solubility of the drugs.

Unpublished international patent application no PCT/AU00/00452 teaches the formation of phosphorylated complex alcohols in conditions which preserve the complex alcohols. These complex alcohols include hormones, phytosterols, tocopherols (chromans), vitamin K1 and other oil-soluble vitamins and dietary supplements as well as drug compounds such as amoxycillin. These phosphorylated complex alcohols are more water soluble than the complex alcohols themselves, but it is desirable to achieve a yet higher level of bioavailability.

In summary, effective delivery of poorly water soluble compounds should not only provide delivery to the intestinal wall but also promote transport through it. There is need for a drug delivery system that embraces these concepts.

Whilst the following discussion concerns tocopherol, it is also to be understood that the same principles apply to any drug hydroxy compounds.

Tocopherol

Vitamin E (tocopherol) is an essential part of skin dynamics and is known to be very important for skin health, with deficiency manifesting as a cornified, scaly delicate skin, thickened epidermis, scaling, lesions, chronic infection, inflammation and erythema. Vitamin E is the main naturally occurring lipid soluble agent protecting the skin from stress, and is the main lipid soluble agent protecting the cell membrane lipids from peroxidation.

Skin is subject to constant stress due to exposure to everyday elements—sun, wind and water. As a result, it is common for many cosmetic products such as lotions, moisturizers, shampoo and conditioners to contain vitamin E to assist in maintaining skin health and/or mitigate and/or prevent hair and skin damage resulting from ultraviolet radiation and other environmentally produced free radicals. In order to assist in maintaining skin health, it is necessary for the vitamin E to reach the target area of the dermis. The most direct method of achieving this targeting is to apply a topical formulation to the affected area. However, topical application of vitamin E to the skin using current formulations has variable success due to the skin's ability to erect an impenetrable barrier to many outside elements. It is critical to provide for the penetration of vitamin E through the epidermis to the dermis. It is believed that topical formulations using tocopherol acetate have not been able to deliver adequate tocopherol beyond the epidermal layers, and therefore provide little benefit. Since tocopheryl acetate is a lipidic material requiring formulation with an oil in water emulsion, absorption from such a formulation is less than optimal.

The more bioactive salts of tocopheryl phosphate are beginning to also be used by cosmetic formulators. The product produced by known phosphorylation processes is a mixture of mono-tocopheryl phosphate (TP), di-tocopheryl phosphate (T2P), mono-tocopheryl di-phosphate (TP2) and di-tocopheryl pyrophosphate (T2P2). TP is the desired product of known phosphorylation processes as it is hydrophilic. Some unreacted tocopherol (T) is also formed when T2P, TP2 and T2P2 are hydrolyzed to produce more of the desired hydrophilic component TP.

Before the mixture may be used in cosmetic applications, the water solubility must be increased. T2P has poor water solubility and is therefore removed or modified in the prior art. This is time consuming, costly and, unless a proper solvent is chosen, can result in undesirable solvent residues.

Formulation Properties

Cosmetic products must also be aesthetic and pleasant to use. Of course, the products must be compatible with eye, skin and oral mucosa and have an overall toxicity profile appropriate for topical application. Applications which are designed for the oral mucosa and/or lip care must also be of an acceptable taste. If tocopheryl phosphates are to be used as a source of Vitamin E in foaming and cleansing products, then the hydrophobic substances need to be removed or modified to mitigate their foam suppression properties. Consumers have started to prefer transparent creams, lotions and gel vehicles for use on skin and hair, particularly for infant care, as this is a symbol of purity and mildness. Current tocopheryl phosphates cannot be used in such transparent products because they have limited water solubility and form opaque emulsions.

Finally, the opaque creams and lotions made with current tocopheryl phosphate mixtures have considerable stability problems at elevated temperatures and temperatures below freezing because of the limited water solubility of the tocopheryl phosphates.

There is thus a need for a drug delivery system which provides improved bioavailability and/or improved formulation properties.

### SUMMARY OF THE INVENTION

In this specification, the term “hydroxylated active” refers to chemical substances having hydroxy groups which may be phosphorylated and (in the non-phosphorylated form) have a desired activity. The term “hydroxylated active” includes, but is not limited to, drugs, vitamins, phytochemicals, cosmeceuticals, nutraceuticals and other health supplements. The hydroxylated active may be administered through oral, topical, inhalation, opthalmic, intravenous, enteral, parenteral or other appropriate presentations including those commercially utilized.

The present invention relates to the discovery that the reaction product of one or more phosphate derivatives of a hydroxylated active and a complexing agent selected from amphoteric surfactants, cationic surfactants, amino acids having nitrogen functional groups and proteins rich in these amino acids has useful properties.

According to the invention there is provided a composition comprising the reaction product of:

(a) one or more phosphate derivatives of one or more hydroxylated actives; and

(b) one or more complexing agents selected from the group consisting of amphoteric surfactants, cationic surfactants, amino acids having nitrogen functional groups and proteins rich in these amino acids.

Preferably, the mole ratio of phosphate derivatives of one or more hydroxylated actives to complexing agents is in the range of from 1:10 to 10:1. Preferably, the mole ratio of phosphate derivatives of one or more hydroxylated actives to complexing agents is in the range of from 1:2 to 2:1. A person skilled in the art will understand that the resultant composition will be a mixture of complexed and non-complexed phosphate derivatives of hydroxylated actives depending on the amount of complexing agent used.

In a preferred embodiment there is provided a therapeutic formulation comprising (i) the reaction product of (a) and (b); and (ii) an acceptable carrier.

According to a second aspect of the invention, there is provided a method for improving the bioavailability of a hydroxylated active comprising the step of reacting:

(a) one or more phosphate derivatives of one or more hydroxylated actives; with

(b) one or more complexing agents selected from the group consisting of amphoteric surfactants, cationic surfactants, amino acids having nitrogen functional groups and proteins rich in these amino acids.

Preferably, there is a further step of adding an acceptable carrier.

There is also provided a method for administering to a subject a therapeutic formulation with an effective amount of one or more hydroxylated actives comprising administering to the subject a therapeutic formulation comprising:

(a) an effective amount of the reaction product of:

(i) one or more phosphate derivatives of one or more hydroxylated actives; and

(ii) one or more complexing agents selected from the group consisting of amphoteric surfactants, cationic surfactants, amino acids having nitrogen functional groups and proteins rich in these amino acids; and

(b) an acceptable carrier.

The complexing agents increase the hydrophilic region on the hydroxylated active to one that is of relatively high electronic charge and attractive to water molecules (more water soluble) which may cause the resulting complexes to be more bioavailable than the parent hydroxylated active. This is possible due to delivery of a complex in the proximity of the intestinal wall in a derivative form which may result in efficient transport and higher tissue penetration. Further, the new complexes are weakly dissociated by water back to the original components of the complex thus releasing the drug, and the process does not require enzyme action or any other reaction to release the hydroxylated active.

Complexation acts to convert lipids to surfactants allowing better emulsification of the active compound. There are a number of situations where complexation may be of value in the drug industry. Complexation may allow conversion of some injectable only formulations to orally available products by improving solubility. Complexation may also decrease injection time, increase predictability of bioavailability and allow further development of compounds whose low bioavailability has previously restricted clinical use.

In a preferred embodiment, the one or more hydroxylated actives are electron transfer agents. Preferably, one of the electron transfer agents is tocopherol. It has been found that complexes of tocopheryl phosphates can be formed which are more soluble in water than the parent tocopheryl phosphates. Further, it is not necessary to remove any T2P prior to forming these complexes. As these complexes of tocopheryl phosphate are more hydrophilic, they are useful for cosmetic formulations. Phosphorylated tocopherol complexed with a tertiary amine acts as both a surfactant and active source of vitamin E, achieving higher bioavailability by quickly reaching the rate limiting CMC because of its higher water solubility or ability to form better emulsions and eventually chylomicrons if used in an oral or injectable formulation.

### DETAILED DESCRIPTION

The following terms are used throughout the specification and are intended to have the following meanings:

The term “hydroxylated active” as defined above. Examples of hydroxylated actives include but are not limited to:

1. electron transfer agents (as defined below)

2. narcotic analgesics such as morphine and levorphanol,

3. non narcotic analgesics such as codeine and acetaminophen,

4. corticosteroids such as cortisone,

5. anaesthetics such as propofol,

6. antiemetics such scopolamine,

7. sympathomimetic drugs such as adrenaline and dopamine,

8. antiepileptic drugs such as fosphenytoin,

9. anti-inflammatory drugs such as ibuprofen,

10. thyroid hormones and antithyroid drugs including thyroxine,

11. phytochemicals including α-bisabolol, eugenol, silybin, soy isoflavones,

12. iridoid gylcosides including aucubin and catalpol,

13. sesquiterpene lactones including pseudoguaianolide from Arnica chamissonis,

14. terpenes including rosmarinic acid and rosmanol,

15. phenolic glycosides including the salicylates salicin, saligenin and salicyclic acid,

16. triterpenes taxasterol or α-lactucerol, and isolactucerol,

17. p-hydroxyphenylacetic acid derivative taraxacoside,

18. hydroquinone derivatives including arbutin,

19. phenylalkanones including gingerols and shagaols,

20. hypercin, and

21. acylphloroglucides including xanthohumol, lupulone, humulone and 2-methylbut-3-en-2-ol.

The term “electron transfer agent” is used herein to refer to the class of hydroxylated actives which (in the non-phosphorylated form) can accept an electron to generate a relatively stable molecular radical or accept two electrons to allow the compound to participate in a reversible redox system. Examples of classes of electron transfer agents that may be phosphorylated include hydroxy chromans including alpha, beta and gamma tocols (eg tocopherol) and tocotrienols in enantiomeric and raecemic forms; quinols being the reduced forms of vitamin K1 and ubiquinone; hydroxy carotenoids including retinol; calciferol and ascorbic acid.

The term “effective amount” is used herein to refer to an amount that reaches the target site in the human or animal in an amount that is measurably effective in the reduction of one or more symptoms.

The term “acceptable carrier” is used herein to refer to a carrier considered by those skilled in the drug, food or cosmetic arts to be non-toxic when used to treat humans, animals or plant in parenteral or enteral formulations. For example, ingestible compositions may include phospholipids such as lecithin, cephalins and related compounds.

The “phosphate derivatives of hydroxylated actives” comprise compounds covalently bound by means of an oxygen to the phosphorus atom of a phosphate group. The oxygen atom is typically derived from a hydroxyl group on the electron transfer agents. The phosphate derivative may exist in the form of a free phosphate acid, a salt thereof, a di-phosphate ester thereby including two molecules of electron transfer agent, a mixed ester including two different compounds selected from electron transfer agents, a phosphatidyl compound wherein the free phosphate oxygen forms a bond with an alkyl or substituted alkyl group. For example, tocopheryl phosphate may be provided mixed with ascorbyl phosphate or as an ascorbyl/tocopheryl phosphate. Similarly, ascorbyl phosphates may be combined with tocotrienol phosphates and/or ubiquinol phosphates. Similarly, retinyl phosphate could be combined with tocopheryl phosphates and/or ascorbyl phosphates.

Phosphorylation may be accomplished by any suitable method. Preferably, the hydroxyl group in the hydroxylated active is phosphorylated using P₄O₁₀ according to the method in international patent application no PCT/AU00/00452. Excess diphosphate derivatives may be hydrolyzed using methods known to those skilled in the art.

Complexing agents may be selected from alkyl amino/amido betaines, sultaines, phosphobetaines, phosphitaines, imidazolimum and straight chain mono and dicarboxy ampholytes, quaternary ammonium salts, and cationic alkoxylated mono and di-fatty amines, and amino acids having nitrogen functional groups and proteins rich in these amino acids. A preferred complexing agent is N-lauryl imino di-propionate.

The amino acids having nitrogen functional groups include glycine, arginine, lysine and histidine. Proteins rich in these amino acids may also be used as complexing agents, for example, casein. These complexing agents are used when the composition needs to be ingestible.

The amphoteric surfactants may be ampholytic surfactants, that is, they exhibit a pronounced isoelectric point within a specific pH range; or zwitterionic surfactants, that is, they are cationic over the entire pH range and do not usually exhibit a pronounced isoelectric point. Examples of these amphoteric surfactants are tertiary substituted amines, such as those according to the following formula:

NR¹R²R³

wherein R¹ is chosen from the group comprising R⁴ or R⁴CO wherein R⁴ is straight or branched chain mixed alkyl radicals from C6 to C22.

R² and R³ are either both R⁵ or one R⁵ and one H wherein R⁵ is chosen from the group comprising CH₂COOX, CH₂CHOHCH₂SO₃X, CH₂CHOHCH₂OPO₃X, CH₂CH₂COOX, CH₂COOX, CH₂CH₂CHOHCH₂SO₃X or CH₂CH₂CHOHCH₂OPO₃X and X is H, Na, K or alkanolamine.

In addition, when R¹ is RCO then R² may be (CH₃) and R³ may be (CH₂CH₂)N(C₂H₄OH)—H₂CH₂OPO₃Na or R² and R³ together may be N(CH₂)₂N(C₂H₄OH)CH₂COOH.

Commercial examples are DERIPHAT sold by Henkel/Cognis, DEHYTON sold by Henkel/Cognis, TEGOBETAINE sold by Goldschmidt and MIRANOL sold by Rhone Poulenc.

Cationic surfactants, such as quaternary ammonium compounds, will also form complexes with phosphorylated derivatives of drug hydroxy compounds such as tocopheryl phosphates. Examples of cationic surfactants include the following:

(a) RN⁺(CH₃)₃Cl⁻

(b) [R₂N⁺CH₃]₂SO₄²⁻

(c) [RCON(CH₃)CH₂CH₂CH₂N⁺(CH₃)₂C₂H₄OH]₂ SO₄²⁻

(d) Ethomeens: RN[(CH₂CH₂O)ₓ CH₂OH][(CH₂CH₂O)_{y} CH₂OH] wherein x and y are integers from 1 to 50.

wherein R is C8 to C22 straight or branched chain alkyl groups or mixed alkyl groups.

Silicone surfactants including hydrophilic and hydrophobic functionality may also be used, for example, dimethicone PG betaine, amodimethicone or trimethylsilylamodimethicone. For example, ABILE 9950 from Goldschmidt Chemical Co. The hydrophobe can be a C6 to C22 straight or branched alkyl or mixed alkyl including fluoroalkyl, fluorosilicone and or mixtures thereof. The hydrophilic portion can be an alkali metal, alkaline earth or alkanolamine salts of carboxy alkyl groups or sulfoxy alkyl groups, that is sultaines, phosphitaines or phosphobetaines or mixtures thereof.

These complexes may be formed by the reaction of one or more phosphate derivatives of one or more hydroxylated actives and one or more complexing agents selected from the group consisting of amphoteric surfactants and cationic surfactants. Complexes of phosphate derivatives of hydroxylated actives can be made by neutralization of the free phosphoric acid ester directly during manufacture as a raw material suitable for compounding or in-situ blending of the mixed sodium salts with the complexing agents during the finished cosmetic formulation process.

Formulations according to the present invention may contain from about 0.5 to about 30 weight percent hydroxylated active phosphate derivative complexes, preferably from about 1 to about 20 wt percent, more preferably about 2 to about 15 wt percent, and most preferably about 3 to about 12 wt percent, based on the total weight of the composition. A most preferred amount of hydroxylated active phosphate derivative complexes is about 5 to about 10 wt. %.

Complexes of tocopheryl phosphate are particularly preferred electron transfer agent phosphate complexes useful in the present invention. The tocopheryl phosphate product produced by known phosphorylation processes is a mixture of mono-tocopheryl phosphate (TP), di-tocopheryl phosphate (T2P), mono-tocopheryl di-phosphate (TP2) and di-tocopheryl pyrophosphate (T2P2). The preferred result is usually a mixture of about 70/30 TP to T2P, however this results in limited water solubility. Before the mixture may be used in cosmetic applications, the water solubility must be increased by forming complexes according to the invention.

Consumers have started to prefer transparent creams, lotions and gel vehicles for use on skin and hair, particularly for infant care, as this is a symbol of purity and mildness. Tocopheryl phosphates available prior to the present development could not be used in such transparent products because they have limited water solubility and form opaque emulsions. Finally, the opaque creams and lotions made with such prior tocopheryl phosphate mixtures have considerable stability problems at elevated temperatures and temperatures below freezing because of the limited water solubility of the tocopheryl phosphates.

The hydroxylated actives phosphate derivative complexes are water-soluble and thus enhance the incorporation of the hydroxylated actives into water-based drug and cosmetic formulations. The water solubility of the complexes also increases the stability of the formulations over a wide range of temperatures and permits that manufacture of clear or transparent solutions. It has also been found that the complexes have increased surface activity and exhibit good foaming properties. This makes the complexes useful for cosmetic products such as cleansing agents and shampoo. The complexes provide stable cosmetic products, which are consumer acceptable while minimizing the problems with current hydroxylated active formulations.

Hydroxylated actives phosphate derivative complexes may be used in various products including antiperspirant sticks, deodorant sticks, sunscreens, facial cleansers, make-up removers, hair pomades, facial gels, oil in water moisturizers, lotions, conditioners, shampoos, conditioning shampoos, toothpaste, and foaming body washes.

The formulation or method of the invention may also be delivered in any suitable drug delivery system applied to the dermis including patches, gels, depots, plasters, aerosols and other sustained or delayed release systems designed to alter absorption kinetics.

A person skilled in the art will know what components may be used as the acceptable carrier for the compositions of the present invention. These will include excipients such as solvents, surfactants, emollients, preservatives, colorants, fragrances and the like.

There is also provided a method for increasing the water solubility and/or detergent properties of tocopheryl phosphate dervatives comprising the step of reacting phosphorylated tocopherol with one or more complexing agents selected from the group consisting of amphoteric surfactants and cationic surfactants.

### EXAMPLES

The invention will now be further explained and illustrated by reference to the following non-limiting examples.

The following components were used in the examples.

| | |
|---|---|
| Brij 72 | POE 2 Stearyl Ether ex Unichema Americas |
| Brij 721 | POE 21 Stearyl Ether ex ICI or |
| | Uniqema Americas |
| Carbopol 934 25% | Ex BF Goodrich |
| Cetiol LC | Ex Henkel/Cognis |
| Cetiol V | Ex Henkel/Cognis |
| Cetiol 3600 | Ex Henkel/Cognis |
| Citric acid | Ex Henkel/Cognis |
| Cocamide mea | Ex Croda |
| Cocamidopropylbetaine | 35% commercial formulation called |
| | Velvetex BA 35 ex |
| Dehyquart F | cationic conditioner ex Henkel/Cognis |
| Deriphat 160 | a 97% free flowing powder of lauryl-imino- |
| | diproprionate ex Henkel/Cognis |
| Di-sodium-N-lauryl beta | Ex Henkel/Cognis |
| imino dipropionate | |
| Drakeol 9 | LT Mineral Oil ex Penreco |
| Emerest 132 | Stearic Acid ex Cognis |
| Emerest 2400 | Ex Henkel/Cognis |
| Emerest 2314 | Ex Henkel/Cognis |
| Emulgin B2 | Ex Henkel/Cognis |
| Germaben II | Preservative ex Sutton Labs |
| Glycerin | Ex Henkel/Cognis |
| Isostearyl imidazoline | Miranol BM ex Rhone Poulenc |
| Kathon CG | Ex Rohm & Haas |
| Lanette O | Ex Henkel/Cognis |
| Lauramide mea | 100% commercial formulation called |
| | Standamide mea ex Henkel/Cognis |
| Microfine TiO₂ | Ex Tayca Corp |
| Mixed waxes | Carnube, paraffin, beeswax ex Croda |
| Natrasol 250 HHR | Ex Hercules |
| Oils emollients | Ex Croda |
| Pelemol PDD | Propylene Glycol Dicaprylate/Dicaprate |
| | ex Phoenix |
| Peppermint Oil | Ex Firmenich |
| P₄O₁₀ | Ex China |
| Red iron oxide | Ex Warner Jenkinson |
| Silicones | polydimethylsiloxane polymers ex |
| | Dow Corning |
| Sodium lauryl 2 ether | 50% commercial formulation called Standapol |
| sulfate | ES 250 ex Henkel/Cognis |
| Sodium lauryl-3-ether | Ex Henkel/Cognis |
| sulfate | |
| Stearyl alcohol | Ex Croda |
| Tocopherol | Ex Hoffman La-Roche |
| Triethanolamine | Ex Henkel/Cognis |

### Example 1

Complexes of tocopheryl phosphates with ampholytic surfactant are prepared (Complex A).

Tocopherol was treated with P₄O₁₀ as outlined in PCT/AU00/00452 followed by hydrolysis of T₂P₂. The resultant tocopheryl phosphate mixture was reacted with an equimolar amount of di-sodium-N-lauryl beta imino dipropionate. The water content was adjusted to form viscous slurry of about 30-70% wt/wt total solids. The pH was adjusted to 6.0-6.5 using either citric acid or additional beta imino surfactant. The slurry can be dried to the desired active concentration as slurry or as a powder via any conventional drying process i.e. oven-tray-drier and ground via fitzmill to desired particle size. The finished product was a free flowing white to off white powder or aqueous slurry, either of which was dispersible in water.

### Example 2

Complexes of tocopheryl phosphates with a zwitter-ionic surfactant were prepared from sodium salts of tocopheryl phosphates (Complex B). The sodium salts of tocopheryl phosphate, the zwitterionic surfactant and Complex B were tested for foaming properties using the hand lather test.

Part A: Preparation of Sodium Salts of Tocopheryl Phosphates

The tocopherol was treated with P₄O₁₀ as outlined in PCT/AU00/00452 followed by hydrolysis of T₂P₂. After hydrolysis, the tocopheryl phosphates were neutralized to the mono- and di-sodium salts. The resulting product was a viscous tan paste with a Gardiner color of about 8-10 and a pH of 8.0-8.5.

A 2% wt/wt aqueous solution of this paste formed an emulsion with a particle size of at least 10 microns (milky), which produced little or no foam as per hand lathering tests. The emulsion was unstable after two days at 50° C. and after one week at ambient room temperature.

Part B:—Preparation of Complex B

Forty parts of the tocopheryl phosphates paste formed in part A were mixed with 60 parts of cocamidopropylbetaine containing sufficient water to form a 40% wt/wt slurry using a Waring blender. The weight ratio of betaine to tocopheryl phosphate was 1.5:1. The pH was adjusted to 6.0-6.5 using citric acid.

A 5% active solution containing 40% tocopheryl phosphate (equivalent to the 2% wt/wt solution prepared in part A) formed a translucent emulsion with particles of less than 2 microns, which produced copious foam via hand lathering, tests. This foam was denser than the foam produced by either the cocamidopropylbetaine or the tocopheryl phosphates from part A alone. The hand lathering tests showed that a residual amount of the product provided a tactile skin feel—an indication of adherence to skin and keratin fiber.

Properties

| | |
|---|---|
| Appearance | a translucent emulsion |
| pH as is | 6.0-6.5 |
| Lather | Copious foam |
| Stability 50° C. | Stable and clear at least one week |

### Example 3

In this example, complexes were dry blended. Certain complexes can also be dry blended prior to either forming slurry or compounding in-situ.

Forty parts of mixed sodium salts of tocopheryl phosphates were ground to a powder via freeze drying and mixed in a Waring blender with sixty parts of Deriphat 160 a 97% free flowing powder) for twenty minutes to form a homogeneous free flowing powder consisting of di-sodium lauryl-imino-diproprionate tocopheryl phosphate complexes.

### Example 4

In this example, a hand and body wash was formulated using Complex A from Example 1

The tocopheryl phosphate salts were heated with water until clear and homogeneous. Ammonium lauryl sulfate was added and mixed until clear. Cocamide Mea was added and the pH adjusted to 5.5 to 6.0 with citric acid. The solution was cooled to 35° C. and Kathon CG added and mixed for ten minutes. Deionized water was added to complete the finished product to 100 parts total. Sodium chloride was added to adjust viscosity to 4000-5000 centipoises at 25° C.

| Ingredient | % wt/wt |
|---|---|
| Complex A from Example 1 | 10 |
| Ammonium lauryl sulfate 30% | 40 |
| Cocamide mea | 2 |
| Kathon cg | 0.05 |
| NaCl, citric acid, deionized water | qs to 100% |

Properties

| | |
|---|---|
| Viscosity at 25° C. | 4000-5000 |
| pH as is | 5.5 to 6.0 |

### Example 5

A foaming shower gel for skin/hair for sports and chlorine scavenging was formulated using Complex B from Example 2.

Fifteen parts of the 40% Complex B from Example 2 were mixed with fifty parts of water and heated to 50° C. and mixed until clear and homogeneous. Thirty parts of 30% active sodium lauryl-3-ether sulfate were added and mixed until the solution was clear and homogeneous. Three parts of cocamide mea were added and the pH adjusted to 6-6.5 with lactic acid followed by cooling to 35° C. 0.1 parts of preservative kathon cg 0.2 were added followed by deionized water to 100% total to give the following formula:

| Ingredient | % Wt/wt |
|---|---|
| Complex B from Example 2 (40%) | 15 |
| Sodium lauryl-3-ether sulfate | 35 |
| Cocamide mea | 3 |
| Preservative, color, fragrance, deionized | Qs to 100% |
| water | |

Properties

| | |
|---|---|
| Viscosity | 25,000 cps |
| pH as is @ 25° C. | 6.0-6.5 |

The complex can also be made in-situ while compounding the finished cosmetic.

### Example 6

A sports shampoo and shower gel was prepared with in-situ formation of the tocopheryl phosphate complexes.

Sixty parts of deionized water were heated to 60-70° C. followed by the addition of seven parts of 35% cocamidobetaine and mixed until clear. Two parts of mixed sodium salts of tocopheryl phosphate were added and mixed until clear and homogeneous. Twenty-five parts of 50% sodium lauryl 2 ether sulfate were added and mixed until solution was clear.

Three parts of cocamide mea were added and mixed until clear. The pH was adjusted to 5.0-5.5 with citric acid and cooled to 35° C. The preservative, color and fragrance were added and the batch adjusted to 100% with deionized water to provide the following formula.

| Ingredient | % wt/wt |
|---|---|
| Sodium lauryl 2 ether sulfate | 25 |
| Cocamidopropylbetaine | 7 |
| Sodium tocopheryl phosphates | 2 |
| Lauramide mea | 3 |
| Citric acid | Qs |
| Preservative and deionized water | Qs to 100% |

Properties

| | |
|---|---|
| Appearance | clear viscous gel |
| viscosity | 25,000 cps |
| pH as is | 5.0-6.0 |
| Lather | rich lubricious |
| Stability 50° C. | Stable and clear for 2 weeks |
| Freeze/Thaw: 2 cycles | Stable |

The gels of this type often require a rheology modification using semi-synthetic polymers such as cellulosic gums as needed.

### Example 7

An economy conditioning shampoo was prepared from the formulation in Example 6.

The product from Example 6 was diluted with deionized water at a wt/wt ratio of 75 parts of Example 6 to twenty five parts of water to provide a shampoo with a viscosity of 3000 cps at 25° C. The product was clear and stable as per Example 6. The product is high foaming/cleansing with the additional benefit of providing perceived body or fullness to hair.

Applications of the complex salts designed for non-foaming areas such as hair conditioners, body and facial creams, sun, shave and lip products etc can be produced via using a higher alkyl chain as the hydrophobic group on the amphoteric portion of the complex and/or the use of cationic salts such as those used in hair conditioners. These products can be made using any of the above methods of complex formation.

### Example 8

A rinse-off hair conditioner was prepared using tocopheryl phosphates with a cationic surfactant to form a complex.

| Ingredient | % wt/wt |
|---|---|
| Dehyquart F | 2 |
| Tocopheryl phosphates | 2 |
| Stearyl alcohol | 1 |
| Brij 721 | 2 |
| Natrasol 250 HHR | 1 |
| Citric acid | 0.5 |
| Preservative, dye and deionized | Qs to 100% |
| water | |

Properties

| | |
|---|---|
| Appearance | clear viscous gel |
| Viscosity | 5000 cps |
| pH as is | 4-5 |
| Lather | rich lubricious |
| Stability 50° C. | Stable for 2 weeks |
| Freeze/Thaw - 2 cycles | Stable |

### Example 9

A facial anti aging crème was prepared using an isostearyl analogue of imidazoline (amphoteric surfactant).

| | |
|---|---|
| Ingredient | % wt/wt |

Part A

| | |
|---|---|
| Isostearyl imidazoline | 1.0 |
| Emulgin B2 | 1.4 |
| Emerest 2400 | 2.0 |
| Lanette O | 2.0 |
| Emerest 2314 | 5.0 |
| Cetiol LC | 3.5 |
| Cetiol V | 3.5 |
| Cetiol 3600 | 3.0 |

Part B

| | |
|---|---|
| Carbopol 934 (25%) | 10.0 |
| Tocopheryl phosphate | 2.0 |
| Deionized water | 57.6 |
| Glycerin | 5.0 |

Part C

| | |
|---|---|
| Triethanolamine | 0.5 |

Part D

| | |
|---|---|
| Germaben II preservative | 1.0 |

Mix parts A and B in separate vessels and heat to 80° C. Add A to B and mix at 80° C. for 10 minutes. Cool to 60° C. then add C. Cool to 60° C. then add D.

Properties

| | |
|---|---|
| Appearance | stable white creme with pleasant |
| | tactile skin feel |
| Stability 50° C. | Stable for 1 month |
| Freeze/Thaw - 2 cycles | Stable |

### Example 10

A lanolin free lipstick was prepared using the complex in Example 9.

| Ingredient | % wt/wt |
|---|---|
| Isostearyl imidazolinium | 3 |
| tocopheryl phosphate | |
| Mixed waxes | 30 |
| Oils emollients | 45 |
| Red iron oxide | 5 |
| Microfine TiO₂ | 5 |
| Silicones | as to 100% |

Stable lipstick with good pay-off and pleasant taste.

### Example 11

A lotion was prepared as follows. The following ingredients are mixed.

| Ingredient | w/w percent |
|---|---|
| cetyl alcohol | 0.75 |
| C12-15 alcohols benzoate | 5 |
| butylated hydroxyanisole | 0.1 |
| PEG-100 stearate | 0.25 |
| water, deionized or distilled | 70.4 |
| propylene glycol | 3.0 |
| tocopheryl phosphate complex | 10.5 |
| (TPC of Ex. 2) | |
| acetone | 10.0 |

### Example 12

A cream was manufactured by mixing the following ingredients:

| Ingredients | w/w percent |
|---|---|
| Cetyl-stearyl alcohol | 1.25 |
| C12-15 alcohol benzoate | 5 |
| Butylated hydroxyanisole | 0.01 |
| PEG-100 stearate | 0.85 |
| Water, deionized or distilled | 69.1 |
| Propylene glycol | 3 |
| Tocopheryl phosphate complex (TPC of Ex 1) | 10.5 |
| Acetone | 10 |

### Example 13

A gel according to the present invention was prepared by combining the following ingredients.

| Ingredient | w/w percent |
|---|---|
| Water, deionized or distilled | 50.65 |
| Veegum. RTM. (R. T. Vanderbilt Co.) | 1.5 |
| Carboxy vinyl polymer (acid) | 1 |
| Diisopropanolamine | 0.75 |
| Ethyl alcohol, 200° | 30.1 |
| Tocopheryl phosphate complex (TPC of Ex. 1) | 15 |

### Example 14

Fifteen mg of Carbomer (15 mg) was added to distilled water (495 mg) while stirring. Stirring was continued for about 45 minutes. A solution of sodium hydroxide (4.09 mg) in distilled water (4.9 ml) was added and stirring continued for 10 minutes. Ethyl alcohol (150 ml) and methyl salicylate (1 mg) were added to the stirred solution, followed by tocopheryl phosphate complex (50% TP complex of Example 1—50% water) (400 mg), and distilled water (80 ml). The resulting mixture was stirred until a smooth gel was obtained.

### Example 15

The following gel formulation was prepared according to the procedure described in Example 14.

| Ingredient | w/w percent |
|---|---|
| tocopheryl phosphate complex | 20 |
| tetracycline | 2 |
| ethyl alcohol | 20 |
| PEG-8 caprate | 6 |
| colloidal mg aluminum silicate | 2.5 |
| hydroxyethylmethylcellulose | 0.75 |
| citric acid | 0.05 |
| water | Q.S. |

### Example 16

Aqueous gel compositions were prepared according to the following formulation:

| Ingredient | w/w percent |
|---|---|
| tocopheryl phosphate complex | 15 |
| retin A | 0.5 |
| carbomer. RTM. 940 | 1 |
| sodium hydroxide to desired pH | |
| water | QS |

### Example 17

A lotion with sunscreen was prepared as follows.

| | Ingredients | % w/w |
|---|---|---|
| A | Brij 72 (POE 2 Stearyl Ether) | 0.5 |
| | Emerest 132 (Stearic Acid) | 2.0 |
| | Pelemol PDD (Propylene Glycol Dicaprylate/ | 10.0 |
| | Dicaprate) | |
| | Drakeol 9 (LT Mineral Oil) | 9.0 |
| | Brij 721 (POE 21 Stearyl Ether) | 1.0 |
| | Octylmethoxy Cinnamate | 7.0 |
| | Benzophenane-3 | 2.0 |
| | Dicorning 200 Fluid (Dimethicane) | 1.0 |
| | Propyl Paraben | 0.1 |
| B | Cabopol Ultrez 10 Slurry 3% | 5.0 |
| | Water | 10.0 |
| C | TEA 99% | 1.2 |
| | Water Distilled | 10.0 |
| | Methyl Paraben | 0.25 |
| | Lauryl Imino Dipropionic Acid Tocopheryl | 7.5 |
| | Phosphate - 40% with DMDMH | |
| | Water Distilled q.s. | 33.45 |

Heat A and C separately to 80° C. Add A to C while mixing with an homogenizer for 2 to 3 min. Remove the mixture from the homogenizer, add B (which has been heated to 70° C.) and then cool to room temperature.

### Example 18

A toothpaste was prepared as follows:

| | Ingredients | % w/w |
|---|---|---|
| A | Sorbitol USP | 15.0 |
| | 40% Lauryl Imino Dipropionic Acid | 7.5 |
| | Tocopheryl Phosphate | |
| B | Glycerin USP 96% | 10.0 |
| | Triclosan | 0.3 |
| | Na-Saccharin USP 40/60 Mesh | 0.2 |
| | Veegum D-Granular | 2.0 |
| | Peppermint Oil | 1.1 |
| | Stepanol WA/100 (Na-Lauryl Sulfate) | 2.2 |
| C | Veegum HF-6% (Ag/Al Silicate) | 16.64 |
| | Blue #1 FD + C (0.6%) | 0.06 |
| D | Na-CMC 7 H 5% | 45.0 |

Mix together the components of A, then add all items of B to A and mix until uniform. Add C and mix until uniform. Finally, add D slowly mixing until uniform.

### Example 19

A tocopheryl phosphate amphoteric complex formulation is prepared as follows:

| Ingredient | % w/w |
|---|---|
| di-sodium alpha tocopheryl phosphate N-lauryl imino | 30% |
| dipropionate complex | |
| Water | 67% |
| Lanolin creme | 3% |

### Example 20

Di-sodium alpha tocopheryl phosphate N-lauryl imino dipropionate complex (a 60/40-N-lauryl imino dipropionate/mixed-phosphate weight ratio) was analyzed in tests as follows.

³¹P NMR

³¹P spectra were carried out at ambient temperature using a Bruker DPX300 spectrometer.

The complex mixture was dissolved in CDCl₃. The spectrum had a single peak at −2.9 ppm and a single peak at −7.9 ppm. There was also a small peak for inorganic phosphates at 1.0 ppm.

The spectrum for pure di-sodium mono-tocopheryl phosphate (dissolved in THF/H₂O (2:1)) consisted of a single peak at 1.1 ppm. The spectrum for pure sodium di-tocopheryl phosphate (dissolved in THF/H20 (2:1)) consisted of a single peak at −7.5.

From this information it can be concluded that a mono-tocopheryl phosphate N-lauryl imino dipropionate complex formed and corresponds to the peak at −2.9 ppm.

Electrospray Mass Spectrometry

The complex product was then analysed by electrospray mass spectrometry on a Micromass Platform II spectrometer using an accelerating voltage of 40V. The spectrum had peaks at 328 for N-lauryl imino dipropionate, 509 for mono-tocopheryl phosphate, 838 for mono-tocopheryl phosphate N-lauryl imino dipropionate complex and 922 for di-tocopheryl phosphate.

The mono-tocopheryl phosphate N-lauryl imino dipropionate complex survived the intense accelerating field. A typical salt would dissociate in such an electron field therefore it is apparent that mono-tocopheryl phosphate N-lauryl imino dipropionate complex is not a typical salt.

Osmometry

A vapour pressure osmometer was used to investigate the dissociation of the di-sodium alpha tocopheryl phosphate N-lauryl imino dipropionate complex by comparing the lowering of the equilibrium temperature to give an identical partial pressure of water vapour around a drop of pure water versus various solutions as an indication of the relative moles of solute. The instrument does not output absolute temperature but instead gives an arbitrary scale that is directly related to sodium chloride as a solute, thus for 0.1M sodium chloride the output was a 29 unit effect.

N-lauryl imino dipropionate alone gives three ions and at 0.05M the effect was 38 units. If the complex was readily dissociated, then the additional tocopheryl phosphate would be expected to increase the effect in the ratio 3:5 by the addition of the charged amino group as a cation and tocopheryl hydrogen phosphate anion. However, addition of 0.05M of tocopheryl phosphate to the 0.05M N-lauryl imino dipropionate resulted in a solution with 36 units.

This result demonstrates that the complex is not ionised in water therefore the complex was not a typical salt where the ionic bonds are readily broken by high dielectric solvents such as water. The behaviour of the complex resembles potassium ferricyanide where the ferricyanide ion is not deemed to be a salt because the iron-cyanide bond is not broken by water as a solvent, such ions are called complexes.

### Example 21

Di-sodium tocopheryl phosphate (1.3 g) was dissolved in 2 ml of water. Arginine hydrochloride (0.5 g) was added and the mixture was intimately mixed for one hour. The mixture increased in viscosity until a gel was formed indicating that a reaction had occurred.

The complex product was then analysed by electrospray mass spectrometry on a Micromass Platform II spectrometer using an accelerating voltage of 40V. The spectrum showed peaks at 510 (tocopheryl phosphate) and 683 (tocopheryl phosphate arginine complex) mass units. The 683 peak indicates the bond between arginine and tocopheryl phosphate survived the intense accelerating field and thus is very strong. A typical salt would not have survived such a field.

### Example 22

Amoxycillin was treated with P₄O₁₀ as outlined in PCT/AU00/00452 to prepare its phosphate derivatives. 445.4 g (1 mole) of amoxycillin phosphoric acid was dispersed in 2 L of water and 327.6 g of Deriphat added and mixed for 10 minutes to generate the complex. The solution was then dried to give the complex. The complex was shown to be readily soluble in water.

### Example 23

Timolol eye drops are utilized to decrease aqueous secretion from the ciliary epithelium and alleviate symptoms of open-angle glaucoma. Sterile opthalmic drops containing 2.5 mg/ml of timolol can be mixed with 3 mg/ml hypromellose solution to reduce “stinging” sensation and improve product absorption.

When 30 mg of timolol is mixed with phosphoric acid and excess fatty acid in sterile water, timolol phosphate is formed. Deriphat was added in an amount equimolar to the timolol phosphate was added and mixed for 10 minutes to form a complex which is more water soluble than the timolol hypromellose solution.

### Example 24

Di-sodium ubiquinyl phosphate (0.3 g) was dissolved in 2 ml of water. Deriphat (0.14 g) was dissolved in 2 ml water and then added to the ubiquinyl phosphate mixture and intimately mixed for one hour. The mixture increased in viscosity until a gel formed indicating that a reaction had occurred.

The product was analyzed by electrospray mass spectrometry on a Micromass Platform II spectrometer using an accelerating voltage of 40V. The spectrum showed peaks at 945 (ubiquinyl phosphate) and 1273 (ubiquinyl phosphate N-lauryl imino dipropionate complex). The 1273 peak indicates the bond between N-lauryl imino dipropionate and ubiquinyl phosphate survived the intense accelerating field and thus is very strong. A typical salt would not have survived such a field.

### Example 25

The skin penetration properties of complexed and non-complexed tocopheryl phosphate (non-complexed (sodium salts)) were compared relative to tocopheryl acetate.

Test Formulations

The test materials were made up on the basis of 5% mixed actives tocopherol (T), tocopheryl phosphate (TP) and tocopheryl diphosphate (T2P) or tocopheryl acetate in a vehicle consisting of 95/5 distilled water/ethanol with pH adjusted (if necessary to 6.5-7.0 with citric acid or dilute NAOH).

Tocopheryl Phosphate Complexes (TPC)

The TPC used was lauryl-imino di-propionic acid tocopheryl phosphate; a surface-active amphoteric phosphate ester complex formed from lauryl imino propionic acid (Deriphat 160) and tocopheryl phosphates.

| | TPC |
|---|---|
| Active | (micrograms per applied dose) |
| Tocopheryl phosphate | 188 |
| di-tocopheryl phosphate | 713 |
| Tocopherol | 20 |

The solution for TPC was based on 40% active mixed phosphates as the latter was reacted/combined in a 60/40-amphoteric/mixed-phosphate weight ratio (1.9-1 mole ratio). 12.5 w/w % of TPC was dissolved in 87.5 w/w % of the 95/5 water/ethanol mixture.

Di-Sodium Salt of Mono and Di-Tocopheryl Phosphates (DSS)

DSS was similar in TP and T2P content, however, unlike TPC, DSS existed as the mixed sodium salts. A slurry of 6.25 w/w % of 80% DSS in 93.75 w/w % of the 95/5-water/ethanol mixture iwa prepared.

| | DSS |
|---|---|
| Active | (micrograms per applied dose) |
| tocopheryl phosphate | 252 |
| di-tocopheryl phosphate | 1194 |
| tocopherol | 24 |

Tocopheryl Acetate (TA)

Tocopheryl acetate was obtained from Roche/BASF. 5.0 w/w % of TA was dispersed in 95.0 w/w % of 95/5 water/ethanol mixture.

Method

The test formulations were evaluated in in vitro human skin penetration studies. Samples were analyzed for the mono- and di-tocopheryl phosphates, free alpha-tocopherol, and tocopheryl acetate by high performance liquid chromatography (HPLC). The tests were conducted by DermTech International (San Diego, Calif.). Human cadaver skin was obtained and prepared. Each formulation was evaluated on triplicate sections from each donor at a topically applied dose of 5 μL/cm². Receptor solutions were collected over 48 hours at pre-selected time intervals. After 48 hours the skin surface was washed with isopropyl alcohol, and the skin was collected and split into epidermis and dermis. The skin sections were extracted with isopropyl alcohol. All collected samples were processed and assayed for tocopherol, tocopheryl acetate, tocopheryl phosphate and di-tocopheryl phosphate.

Mass balance from the samples is between 80-120% of the applied dose.

No tocopherols were observed in the receptor solution. This could be a result of amounts being below limits of detection, or degradation of the various tocopherol species into other, as yet uncharacterized, compounds.

TABLE 1
Skin Penetration Study
Percent Distribution of Tocopherols
Recovered across Samples wt/wt %

| | T | TP | T2P |
|---|---|---|---|
| DSS | | | |
| Surface Wash | 65.05 | 41.40 | 56.05 |
| Epidermis | 26.74 | 47.06 | 37.31 |
| Dermis | 8.24 | 11.42 | 6.62 |
| Dermis/Epidermis Ratio | 0.31 | 0.24 | 0.18 |
| TPC | | | |
| Surface Wash | 50.00 | 48.82 | 70.92 |
| Epidermis | 35.99 | 24.55 | 16.67 |
| Dermis | 14.07 | 26.62 | 12.36 |
| Dermis/Epidermis Ratio | 0.39 | 1.08 | 0.74 |

| TA | Tocopheryl Acetate |
|---|---|
| Surface Wash | 91.48 |
| Epidermis | 7.13 |
| Dermis | 1.39 |
| Dermis/Epidermis Ratio | 0.20 |

Summary of Results

(a) The T, TP and T2P in the DSS and TPC formulations penetrate into the skin more effectively than TA.

(b) TPC is a better delivery system than DSS as shown by a higher TP penetration ratio into the dermis/epidermis.

(c) The enhanced penetration of the tocopheryl phosphates from TPC is most likely the result of the TPC surface-active properties. The TPC is more effective in lowering the surface tension at the liquid/skin interface compared to both DSS and TA. The latter is the most hydrophobic of the three test materials and forms a poor dispersion in the water/alcohol vehicle.

### Example 26

Morphine hydrochloride 32 g (0.1M) and 37.2 g of sodium valerate (0.3M) were dissolved in 100 ml toluene. 12.6 g (0.05M) of P₄O₁₀ was added and mixed with high shear mixing for one hour slowly raising the temperature to 80° C. 1,2-distearoyl glycerol 30 g was added and the high sheer mixing continued for a further hour at 60° C. 100 ml of a 0.5M sodium hydroxide solution was added and the mixture gently stirred then centrifuged and the process repeated. The toluene phase was recovered and washed with 100 ml of 0.1M hydrochloric acid. The toluene phase was recovered and the toluene and valeric acid removed under vacuum to give 1,2-distearoyl phosphatidyl morphine. Morphine phosphate was recovered from the aqueous phases.

12 grams (0.03 g/mole) of disodium-N-lauryl beta imino dipropionate were dissolved in 88 grams of distilled water to provide a 12% wt/wt clear solution with pH 12. 11.43 grams (0.03 g/mole) of morphine-3-phosphoric acid ester were slowly added and mixed until uniform. The resulting product was a complex consisting of N-lauryl beta imino dipropionate-morphine (3) phosphate as a 21.03% wt/wt aqueous dispersion. This complex product was formulated via dilution with water preservative buffers together with gelling agents and applied to the skin to elicit transdermal drug delivery.

The complex product may be modified as needed by increasing or decreasing the molar ratio of the disodium-N-lauryl beta imino dipropionate.

### Example 27

951 g (1 g/mole) of the phosphoric acid ester of Taxol (Paclitaxel) (C₄₇H₅₃NPO₁₈) were complexed with 202 g of lauryl-imino-dipropionate (0.5 g/mole) in 1200 g of deionized water to yield a 49% wt/wt slurry with a pH of 7.5-8.5. Final pH was modified by adding incremental amounts of lauryl-imino-dipropionate.

### Example 28

174 g (1 g/mole) of arginine was added to 1000 g of deionized water to form a clear solution. 238 g (0.25 g/mole) of the phosphoric ester of Taxol was added slowly to form a complex which was 29-30% wt/wt active with a pH of 5-6. The pH was adjusted as desired via adding incremental amounts of arginine or the phosphoric acid ester of Taxol.

### Example 29

860 g (2 g/mole) of the phosphoric acid ester of Alfaxalone (C₂₁H₃₄PO₇) was added to 242.4 g (0.6 g/mole) of disodium lauryl-imino-dipropionate in 2000 ml of deionized water and mixed until homogeneous. The resulting composition is 35-36% solids and had a pH of 4.5-5.5.

### Example 30

174 g (1 g/mole) of arginine was dissolved in 1000 ml of deionized water and mixed until homogeneous. 430 grams (1 g/mole) of the phosphoric acid ester of Alfaxalone was slowly added with mixing followed by the addition of 500 ml of deionized water to yield a 28-29% active complex with a pH of 6.5-7.5.

### Example 31

The free acid of atorvastatin 55.8 g (0.1 M) and 37.2 g of sodium valerate (0.3M) were dissolved in 100 ml toluene. 12.6 g (0.05M) of P₄O₁₀ was added and mixed with high shear mixing for one hour slowly raising the temperature to 80° C. 10 ml of water was added and the high sheer mixing continued for a further hour at 60° C. 100 ml of a 0.1M sodium carbonate solution was added and the mixture gently stirred then centrifuged and the process repeated. The toluene phase was recovered and washed with 100 ml of 0.1 M hydrochloric acid. The toluene phase was recovered and the toluene and valeric acid removed under vacuum to give the phosphoric ester of atorvastatin ([R—(R*,R*)]-2-(4-fluorophenyl)-β-phosphono-δ-hydroxy-5-(1-methylethyl)-3-phenyl-4-[(phenylamino)cabonyl]-1H-pyrrole-1-heptanoic acid).

Complex with Arginine

0.1M of the phosphoric acid ester of atorvastatin was mixed with 0.1M of arginine in 200 ml of water (equimolar proportions). The mixture was mixed thoroughly with good agitation. The final pH was adjusted as desired using small amounts of either component. The mixture was then freeze dried to give the solid complex as a powder.

Complex with lauryl-imino-dipropionate

0.1M of the phosphoric acid ester of atorvastatin was mixed with 0.1M of lauryl-imino-dipropionate in 200 ml of water (equimolar proportions). The mixture was mixed thoroughly with good agitation. The final pH was adjusted as desired using small amounts of either component. The mixture was then freeze dried to give the solid complex as a powder.

### Example 32

The free acid of pravastatin 42.5 g (0.1M) and 37.2 g of sodium valerate (0.3M) were dissolved in 100 ml toluene. 12.6 g (0.05M) of P₄O₁₀ was added and mixed with high shear mixing for one hour slowly raising the temperature to 80° C. 10 ml of water was added and the high sheer mixing continued for a further hour at 60° C. 100 ml of a 0.1M sodium carbonate solution was added and the mixture gently stirred then centrifuged and the process repeated. The toluene phase was recovered and washed with 100 ml of 0.1M hydrochloric acid. The toluene phase was recovered and the toluene and valeric acid removed under vacuum to give the phosphoric ester of pravastatin ([1S-[1α(βS*,δS*),2α,6α,8β(R*),8aα]]-1,2,6,7,8,8a-hexahydro-β-phosphono-δ,6-dihydroxy-2-methyl-8-(2-methyl-1-oxobutoxy)-1-naphthleneheptanoic acid).

Complex with Arginine

50.45 g (0.1M) of the phosphoric acid ester of pravastatin was mixed with 17.4 g (0.1M) of arginine in 200 ml of water (equimolar proportions). The mixture was mixed thoroughly with good agitation. The final pH was adjusted as desired using small amounts of either component. The mixture was then freeze dried to give the solid complex as a powder.

Complex with lauryl-imino-dipropionate

50.45 (0.1M) of the phosphoric acid ester of pravastatin was mixed with 40.4 (0.1 M) of lauryl-imino-dipropionate in 200 ml of water (equimolar proportions). The mixture was mixed thoroughly with good agitation. The final pH was adjusted as desired using small amounts of either component. The mixture was then freeze dried to give the solid complex as a powder.

### Example 33

The free acid of venlafaxine 27.7 g (0.1M) and 37.2 g of sodium valerate (0.3M) were dissolved in 100 ml toluene. 12.6 g (0.05M) of P₄O₁₀ was added and mixed with high shear mixing for one hour slowly raising the temperature to 80° C. 10 ml of water was added and the high sheer mixing continued for a further hour at 60° C. 100 ml of a 0.1M sodium carbonate solution was added and the mixture gently stirred then centrifuged and the process repeated. The toluene phase was recovered and washed with 100 ml of 0.1M hydrochloric acid. The toluene phase was recovered and the toluene and valeric acid removed under vacuum to give the phosphoric ester of venlafaxine (1-[-(dimethylamino)-1-(4-methoxyphenyl)ethyl]cyclohexyl dihydrogen phosphate).

Complex with Arginine

0.1M of the phosphoric acid ester of venlafaxine was mixed with 0.1M of arginine in 200 ml of water (equimolar proportions). The mixture was mixed thoroughly with good agitation. The final pH was adjusted as desired using small amounts of either component. The mixture was then freeze dried to give the solid complex as a powder.

Complex with lauryl-imino-dipropionate

0.1M of the phosphoric acid ester of venlafaxine was mixed with 0.1M of lauryl-imino-dipropionate in 200 ml of water (equimolar proportions). The mixture was mixed thoroughly with good agitation. The final pH was adjusted as desired using small amounts of either component. The mixture was then freeze dried to give the solid complex as a powder.

### Example 34

The free acid of atorvastatin 55.8 g (0.1M) and 37.2 g of sodium valerate (0.3M) were dissolved in 100 ml toluene. 12.6 g (0.05M) of P₄O₁₀ was added and mixed with high shear mixing for one hour slowly raising the temperature to 80° C. 1,2-distearoyl glycerol 30 g was added and the high sheer mixing continued for a further hour at 60° C. 100 ml of a 0.5M sodium hydroxide solution was added and the mixture gently stirred then centrifuged and the process repeated. The toluene phase was recovered and washed with 100 ml of 0.1M hydrochloric acid. The toluene phase was recovered and the toluene and valeric acid removed under vacuum to give 1,2-distearoyl phosphatidyl atorvastatin.

Complex with Arginine

0.1M of 1,2-distearoyl phosphatidyl atorvastatin was mixed with 0.1M of arginine in 200 ml of water (equimolar proportions). The mixture was mixed thoroughly with good agitation. The final pH was adjusted as desired using small amounts of either component. The mixture was then freeze dried to give the solid complex as a powder.

Complex with lauryl-imino-dipropionate

0.1M of 1,2-distearoyl phosphatidyl atorvastatin was mixed with 0.1M of lauryl-imino-dipropionate in 200 ml of water (equimolar proportions). The mixture was mixed thoroughly with good agitation. The final pH was adjusted as desired using small amounts of either component. The mixture was then freeze dried to give the solid complex as a powder.

### Example 35

The free acid of pravastatin 42.5 g (0.1M) and 37.2 g of sodium valerate (0.3M) were dissolved in 100 ml toluene. 12.6 g (0.05M) of P₄O₁₀ was added and mixed with high shear mixing for one hour slowly raising the temperature to 80° C. 1,2-distearoyl glycerol 30 g was added and the high sheer mixing continued for a further hour at 60° C. 100 ml of a 0.5M sodium hydroxide solution was added and the mixture gently stirred then centrifuged and the process repeated. The toluene phase was recovered and washed with 100 ml of 0.1M hydrochloric acid. The toluene phase was recovered and the toluene and valeric acid removed under vacuum to give 1,2-distearoyl phosphatidyl pravastatin.

Complex with Arginine

0.1M of 1,2-distearoyl phosphatidyl pravastatin was mixed with 0.1M of arginine in 200 ml of water (equimolar proportions). The mixture was mixed thoroughly with good agitation. The final pH was adjusted as desired using small amounts of either component. The mixture was then freeze dried to give the solid complex as a powder.

Complex with lauryl-imino-dipropionate

0.1 M of 1,2-distearoyl phosphatidyl pravastatin was mixed with 0.1M of lauryl-imino-dipropionate in 200 ml of water (equimolar proportions). The mixture was mixed thoroughly with good agitation. The final pH was adjusted as desired using small amounts of either component. The mixture was then freeze dried to give the solid complex as a powder.

### Example 36

The free acid of venlafaxine 27.7 g (0.1M) and 37.2 g of sodium valerate (0.3M) were dissolved in 100 ml toluene. 12.6 g (0.05M) of P₄O₁₀ was added and mixed with high shear mixing for one hour slowly raising the temperature to 80° C. 1,2-distearoyl glycerol 30 g was added and the high sheer mixing continued for a further hour at 60C. 100 ml of a 0.5M sodium hydroxide solution was added and the mixture gently stirred then centrifuged and the process repeated. The toluene phase was recovered and washed with 100 ml of 0.1M hydrochloric acid. The toluene phase was recovered and the toluene and valeric acid removed under vacuum to give 1,2-distearoyl phosphatidyl venlafaxine.

Complex with lauryl-imino-dipropionate

88.4 g (0.1M) of venlafaxine phosphatide was mixed with 40.4 g (0.1M) of lauryl-imino-dipropionate in 200 ml of water (equimolar proportions). The mixture was mixed thoroughly with good agitation. The final pH was adjusted as desired using small amounts of either component. The mixture was then freeze dried to give the solid phospatidyl venlafaxine deriphat complex as a powder.

Complex with Arginine

88.4 g (0.1M) of venlafaxine phosphatide was mixed with 17.4 g (0.1M) of arginine in 200 ml of water (equimolar proportions). The mixture was mixed thoroughly with good agitation. The final pH was adjusted as desired using small amounts of either component. The mixture was then freeze dried to give the solid phosphatidyl venlafaxine arginine complex as a powder.

### Example 37

319 grams (1M) of the phosphoric acid ester of acyclovir was mixed with 202 grams (0.5M) of lauryl imino disodium propionate (trade name DERIPHAT 160) in 800 grams of deionized water (20.2% wt/wt solution) at ambient conditions until homogeneous. The resulting complex was a 39.44% solution with pH 6-7.

### Example 38

319 grams (1M) of the phosphoric acid ester of acyclovir was complexed with 174 grams (1M) of arginine in 800 grams of deionized water. The resulting solution was 38.13% wt/wt and had a pH of 6-9.

### Example 39

546 grams (1.5M) of the phosphoric acid ester of AZT (3′azido-azidothymidenyl phosphate) was mixed with 202 grams (0.5M) of lauryl-imino-dipropionate in 800 grams of deionized water to form the soluble complex with 48.32% solids and a pH of 5-6.

### Example 40

728 grams (2M) of the phosphoric acid ester of AZT was complexed with 131 grams (0.75M) of arginine in 1000 grams of deionized water to form the soluble complex with 46.2% active and a pH of 4.0-4.5.

### Example 41

728 grams (2M) of the phosphotidyl ester of acyclovir (1-O-octadecyl-sn-glycero-3-phospho-acyclovir), was complexed with 131 grams (0.75M) of arginine in 1000 grams of deionized water to form the soluble complex which is 46.2% active and has a pH of 4.0-4.5.

### Example 42

An anti-inflammatory mouthwash was prepared as follows:

| Ingredient | % wt/wt |
|---|---|
| Cetyl pyrdinium chloride | 0.2000 |
| Lauryl imino dipropionic acid tocopheryl | 3.330 |
| phosphate | |
| Tween 20 (polysorbate 20) | 2.0000 |
| Poloxymer 407 | 2.0000 |
| Menthol | 0.1000 |
| Potassium sorbate | 2.0000 |
| Propylene glycol | 2.0000 |
| FD&C green #3 | 0.001O |
| FD&C yellow #5 | 0.0005 |
| Flavor mint | 0.2000 |
| Sorbitol | 5.0000 |
| Purified water | QS AD 100% |

Procedure

Charge main vessel with water, sorbitol and propylene glycol and heat to 60° C. with agitation (Vessel #1).

Vessel #2—premix poloxomer, TWEEN 20 and lauryl imino dipropionic acid tocopheryl phosphate and heat to 60° C. and mix until homogeneous.

Add #2 to #1 with mixing and agitate until homogeneous.

Cool to 30° C. and add menthol, dyes and flavor. Add sorbate and cetyl pyridinium chloride.

### Example 43

An anticavity and anti inflammatory mouthwash was prepared as follows.

0.05% sodium flouride (0.0226% fluoride ion) as active ingedient for anti cavity activity was added to the formulation from example 42.

### Example 44

A teething gel for infants for pain mitigation and anti-inflammatory was prepared as follows:

| Ingredient | % wt/wt |
|---|---|
| Benzocaine | 7.5000 |
| lauryl imino dipropionic acid | 7.5000 |
| tocopheryl phosphate | |
| Glycerine | 3.0000 |
| Polyethylene glycols | 10..0000 |
| Sorbitol | 5.0000 |
| Peg 60 castor oil | 3.0000 |
| FD&C red 40 | 0.0001 |
| Sorbic acid | 2.0000 |
| Purified water | QS AD 100% |

Procedure

(1) pre-mix benzocaine with peg glycols and heat to 50° C. until homogeneous

(2) premix lauryl imino dipropionic acid tocopheryl phosphate with PEG 60 castor oil and heat to 50-60° C. Mix until homogeneous.

(3) charge main vessel with water, sorbitol and glycerine and heat to 50-60° C.

add #2 to #3 and mix followed by addition of #1. Mix until homogeneous.

cool to 30° C. and add dye and sorbic acid. Mix until homogeneous.

### Example 45

An anti-inflammatory dental adhesive cream was prepared as follows:

| Ingredient | % wt/wt |
|---|---|
| Mineral oil | 20 |
| White petrolatum | 25 |
| Silicone wax | 21 |
| Ganex v-220 | 5 |
| Lauryl Imino Dipropionic Acid | 10 |
| Tocopheryl Phosphate | |
| Olive oil | 12 |
| Fumed silica | 1 |
| Stearyl alcohol | 3 |
| Peg 60 castor oil | 3 |
| Flavor dye | QS |

Procedure

(1) Charge a Patterson or day type with heated jacket and blade mixer with mineral oil and petrolatum and heat to 60° C. Add silicone wax, olive oil, fumed silica and stearyl alcohol. Mix until homogeneous.

(2) Premix lauryl imino dipropionic acid tocopheryl phosphate and peg castor oil.

Add 2 to 1 while maintaining temp at 60° C. Cool to 40° C. and add flavor and dye.

The word ‘comprising’ and forms of the word ‘comprising’ as used in this description and in the claims does not limit the invention claimed to exclude any variants or additions.

Modifications and improvements to the invention will be readily apparent to those skilled in the art. Such modifications and improvements are intended to be within the scope of this invention.

What is claimed is:

1. A composition comprising the reaction product of:
(a) one or more phosphate derivatives of one or more hydroxylated actives; and
(b) one or more complexing agents selected from the group consisting of amphoteric surfactants, cationic surfactants, amino acids having nitrogen functional groups and proteins rich in these amino acids.

2. A composition according to claim 1 wherein the complexing agents are selected from the group consisting of silicone surfactants, alkyl amino/amido betaines, sultaines, phosphobetaines, phosphitaines, imidazolimum and straight chain mono and dicarboxy ampholytes, quaternary ammonium salts, and cationic alkoxylated mono and di-fatty amines.

3. A composition according to claim 1 wherein the complexing agent is N-lauryl imino di-propionate.

4. A composition according to claim 1 wherein the complexing agents are selected from tertiary substituted amines according to the following formula:
NR¹R²R³
wherein R¹ is selected from the group comprising R⁴ and R⁴CO wherein R⁴ is straight or branched chain mixed alkyl radicals from C6 to C22;
R² and R³ are either both R⁵ or one R⁵ and one H wherein R⁵ is chosen from the group comprising CH₂COOX, CH₂CHOHCH₂SO₃X, CH₂CHOHCH₂OPO₃X, CH₂CH₂COOX, CH₂COOX, CH₂CH₂CHOHCH₂SO₃X or CH₂CH₂CHOHCH₂OPO₃X and X is H, Na, K or alkanolamine; and
wherein when R¹ is R⁴CO then R² may be (CH₃) and R³ may be (CH₂CH₂)N(C₂H₄OH)—H₂CH₂OPO₃Na or R₂ and R₃ together may be N(CH₂)₂N(C₂H₄OH)CH₂COOH.

5. A composition according to claim 1 wherein the cationic surfactants are selected from the group comprising:
(a) RN⁺(CH₃)₃Cl⁻;
(b) [R₂N⁺CH₃]₂SO₄²⁻;
(c) RCON(CH₃)CH₂CH₂CH₂N⁺(CH₃)₂C₂H₄OH]₂SO₄²⁻;
(d) RN[(CH₂CH₂O )ₓCH₂OH][(CH₂CH₂O)_{y}CH₂OH] wherein x and y are integers from 1 to 50; and
wherein R is C8 to C22 straight or branched chain alkyl groups or mixed alkyl groups.

6. A composition according to claim 1 wherein the complexing agent is an amino acid selected from arginine, lysine or histadine.

7. A composition according to claim 1 wherein one or more of the hydroxylated actives is an electron transfer agent.

8. A composition according to claim 7 wherein the electron transfer agent is tocopherol.

9. A composition according to claim 1 wherein there is more than one phosphate derivative of one hydroxylated active.

10. A composition according to claim 1 wherein there is more than one phosphate derivatives of more than one hydroxylated actives.

11. A therapeutic formulation for use on humans, animals or plants comprising:
(a) an effective amount of the reaction product of:
(i) one or more phosphate derivatives of one or more hydroxylated actives; and
(ii) one or more complexing agents selected from the group consisting of amphoteric surfactants, cationic surfactants, amino acids having nitrogen functional groups and proteins rich in these amino acids; and
(b) an acceptable carrier.

12. A method for improving the bioavailability of hydroxylated actives comprising the step of reacting:
(a) one or more phosphate derivatives of one or more hydroxylated actives; with
(b) one or more complexing agents selected from the group consisting of amphoteric surfactants, cationic surfactants, amino acids having nitrogen functional groups and proteins rich in these amino acids.

13. A method according to claim 12 further comprising the step of adding an acceptable carrier.

14. A method for administering to a subject a therapuetic formulation with an effective amount of one or more hydroxylated actives comprising administering to the subject a formulation comprising:
(a) an effective amount of the reaction product of:
(i) one or more phosphate derivatives of one or more hydroxylated actives; and
(ii) one or more complexing agents selected from the group consisting of amphoteric surfactants, cationic surfactants, amino acids having nitrogen functional groups and proteins rich in these amino acids; and
(b) an acceptable carrier.

15. A composition comprising the reaction product of:
(c) one or more phosphate derivatives of tocopherol; and
(d) one or more complexing agents selected from the group consisting of amphoteric surfactants, cationic surfactants, amino acids having nitrogen functional groups and proteins rich in these amino acids.

16. A composition comprising the reaction product of:
(a) one or more phosphate derivatives of one or more hydroxylated actives; and
(b) one or more complexing agents selected from the group consisting of amphoteric surfactants and cationic surfactants.

17. An ingestible composition comprising the reaction product of:
(a) one or more phosphate derivatives of one or more hydroxylated actives; and
(b) one or more complexing agents selected from the group consisting amino acids having nitrogen functional groups and proteins rich in these amino acids.
